(19) United States
(12) Patent Application Publication
(10) Pub. No.: US 2008/0306389 A1
(43) Pub. Date: Dec. 11, 2008

(54) ULTRASONIC ENDOSCOPE AND ULTRASONIC ENDOSCOPIC APPARATUS

(75) Inventors: Kazuhiko NAGANO, Kaisei-machi (JP); Hiroaki Hyuga, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/131,975

(22) Filed: Jun. 3, 2008

(30) Foreign Application Priority Data
Jun. 5, 2007 (JP) 2007-149499

(51) Int. Cl. A61B 8/12

(52) U.S. Cl. 600/462

(57) ABSTRACT

An ultrasonic endoscope having a small size and a slight temperature rise even when the transmission output is increased. The ultrasonic endoscope includes: an ultrasonic transducer part; a Peltier element having a cooling surface coupled to the ultrasonic transducer part and a radiating surface; a flexing part for supporting the ultrasonic transducer part and the Peltier element; a coupling part for coupling the flexing part to an operation part; a covering material for covering the flexing part and the coupling part; a thermal insulating material provided inside of the covering material, for thermally insulating a part from the radiating surface of the Peltier element to the operation part from outside; and a heat-conducting member provided inside of the thermal insulating material and coupled to the radiating surface of the Peltier element, for transferring heat generated in the ultrasonic transducer part to the operation part.

### BACKGROUND OF THE INVENTION

1. Field of the Invention

The present invention relates to an ultrasonic endoscope to be used for body cavity examination of upper digestive organs, bronchial tube, and so on, and the present invention further relates to an ultrasonic endoscopic apparatus including the ultrasonic endoscope.

2. Description of a Related Art

In medical fields, various imaging technologies have been developed in order to observe the interior of an object to be inspected and make diagnoses. Among them, especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but gynecology, circulatory system, digestive system, etc.

The ultrasonic imaging is an image generation technology utilizing the nature of ultrasonic waves that the waves are reflected at a boundary between regions having different acoustic impedances (e.g., a boundary between structures). Typically, an ultrasonic diagnostic apparatus is provided with a body surface ultrasonic probe to be used in contact with the object or an intracavity ultrasonic probe to be used by being inserted into a body cavity of the object. Further, in recent years, an ultrasonic endoscope in combination of an endoscope for optically observing the interior of the object and an intracavity ultrasonic probe has been used.

Ultrasonic beams are transmitted toward the object such as a human body and ultrasonic echoes generated by the object are received by using the ultrasonic endoscope, and thereby, ultrasonic image information is acquired. On the basis of the ultrasonic image information, ultrasonic images of structures (e.g., internal organs, diseased tissues, or the like) existing within the object are displayed on a display unit of an ultrasonic endoscopic apparatus main body connected to the ultrasonic endoscope.

As an ultrasonic transducer for transmitting and receiving ultrasonic waves, a vibrator (piezoelectric vibrator) having electrodes formed on both sides of a material that expresses a piezoelectric property (a piezoelectric material) is generally used. As the piezoelectric material, piezoelectric ceramics represented by PZT (Pb (lead) zirconate titanate), a polymeric piezoelectric material represented by PVDF (polyvinylidene difluoride), or the like is used.

When a voltage is applied to the electrodes of the vibrator, the piezoelectric material expands and contracts due to the piezoelectric effect to generate ultrasonic waves. Accordingly, plural vibrators are one-dimensionally or two-dimensionally arranged and the vibrators are sequentially driven, and thereby, an ultrasonic beam to be transmitted in a desired direction can be formed. Further, the vibrators expand and contract by receiving propagating ultrasonic waves and generate electric signals. These electric signals are used as reception signals of the ultrasonic waves.

When ultrasonic waves are transmitted, drive signals having great energy are supplied to the ultrasonic transducers. In this regard, not the entire energy of the drive signals is converted into acoustic energy but a significant proportion of the energy becomes heat, and there has been a problem that the temperature rises in use of the ultrasonic endoscope. However, the insertion part of the ultrasonic endoscope is used in direct contact with the living body such as a human body, and a request that the surface temperature of the insertion part of the ultrasonic endoscope is controlled to a predetermined temperature or less has been made.

As a related technology, Japanese Patent Application Publication JP-P2005-13461A discloses an ultrasonic probe characterized by having radiating means formed by arranging plural Peltier elements in a cascade with respect to the heat flow from the inside heat generation location to the radiation location to the outside. However, the system disclosed in JP-P2005-13461A uses infrared radiation, and it is necessary to take the area of the radiating surfaces of the Peltier elements larger in order to ensure the heat release between two Peltier elements. Accordingly, the Peltier elements become larger and difficult to be provided within an ultrasonic endoscope. Further, it is difficult to secure the space where plural Peltier elements are provided to enable heat transfer by the heat radiation among the plural Peltier elements.

Further, Japanese Patent Application Publication JP-P2006-204552A discloses an ultrasonic probe in which the heat generated in a vibrator part and a circuit board is transferred to a shield case via a heat-conducting part, and the heat transferred to the shield case is absorbed by a heat absorbing part for cooling the vibrator part. However, JP-P2006-204552A does not disclose cooling of the vibrator part of an ultrasonic endoscope.

In an ultrasonic endoscope to be used by being inserted into a body cavity of a patient, in order to reduce the physical stress on the patient to be examined, it is desired that the diameter of the insertion part is made smaller. Specifically, in body cavity examination of upper digestive organs, bronchial tube, and so on, an endoscope having a small-diameter is used, and downsizing of the ultrasonic endoscope becomes a challenge in view of reduction in physical stress on patients.

Further, in the ultrasonic endoscope, an improvement in diagnostic accuracy by stacking the ultrasonic transducers for higher reception sensitivity is being considered. However, as the transmission output of ultrasonic waves is increased by stacking the ultrasonic transducers, the amount of heat released from the ultrasonic transducers becomes larger. According to the structure of a conventional ultrasonic endoscope, if the transmission output of ultrasonic waves is increased by stacking the ultrasonic transducers, there is a problem that the temperature of the insertion part in contact with the inner wall of the body cavity rises due to the heat generation of the ultrasonic transducers. With downsizing of the ultrasonic endoscope, the temperature rise of the insertion part due to the heat generation of the ultrasonic transducers has become an increasingly serious problem, and the problem is an issue to be solved.

Furthermore, in the case where an imaging device (CCD or the like) and a light guide are attached to the insertion part of the ultrasonic endoscope, a problem of temperature rise in the insertion part due to heat generation from the imaging device and the light guide exit part arises, and the problem is an issue to be solved.

### SUMMARY OF THE INVENTION

The present invention has been achieved in view of the above-mentioned problems. A purpose of the present invention is to provide an ultrasonic endoscope having a small size and a slight temperature rise of an insertion part even when the transmission output of an ultrasonic transducer part is increased or an imaging device is attached thereto, and to provide an ultrasonic endoscopic apparatus including the ultrasonic endoscope.

In order to accomplish the purpose, an ultrasonic endoscope according to one aspect of the present invention includes: an ultrasonic transducer part having plural ultrasonic transducers for transmitting and receiving ultrasonic waves; a Peltier element having a cooling surface and a radiating surface, the cooling surface coupled to the ultrasonic transducer part; a flexing part that is flexible and supports the ultrasonic transducer part and the Peltier element; a coupling part that couples the flexing part to an operation part; a covering material that covers at least the flexing part and the coupling part; a thermal insulating material that is provided inside of the covering material and thermally insulates at least a part from the radiating surface of the Peltier element to the operation part from outside; and a heat-conducting member that is provided inside of the thermal insulating material and coupled to the radiating surface of the Peltier element, and transfers heat generated in the ultrasonic transducer part to the operation part.

Further, an ultrasonic endoscopic apparatus according to one aspect of the present invention includes an ultrasonic endoscope according to the present invention, and an ultrasonic endoscopic apparatus main body that processes signals from the ultrasonic endoscope to display ultrasonic images, and includes a cooing device for cooling the heat-conducting member.

According to the present invention, since the Peltier element is provided in the insertion part of the ultrasonic endoscope, the heat generated in the ultrasonic transducer part is released to the cooling surface of the Peltier element and further released from the radiating surface of the Peltier element to the heat-conducting member provided inside of the covering material for covering the insertion part, and the thermal insulating material is provided between the covering material and the heat-conducting member, an ultrasonic endoscope having a small size and a slight temperature rise of an insertion part even when the transmission output of the ultrasonic transducer part is increased or an imaging device is attached thereto can be provided.

### BRIEF DESCRIPTION OF THE DRAWINGS

FIG. 1 is a schematic diagram showing an appearance of an endoscope according to the respective embodiments of the present invention;

FIG. 2 shows a leading end of the ultrasonic endoscope according to the first embodiment of the present invention;

FIGS. 3A and 3B show a configuration around an ultrasonic transducer part in the insertion part of the ultrasonic endoscope shown in FIG. 2;

FIG. 4 shows an ultrasonic endoscopic apparatus including the ultrasonic endoscope according to the respective embodiments of the present invention and the ultrasonic endoscopic apparatus main body;

FIG. 5 shows a leading end of an ultrasonic endoscope according to the second embodiment of the present invention; and

FIG. 6 shows a leading end of an ultrasonic endoscope according to the third embodiment of the present invention.

### DESCRIPTION OF THE PREFERRED EMBODIMENTS

Hereinafter, embodiments of the present invention will be explained in detail with reference to the drawings. The same reference numbers will be assigned to the same component elements and the description thereof will be omitted.

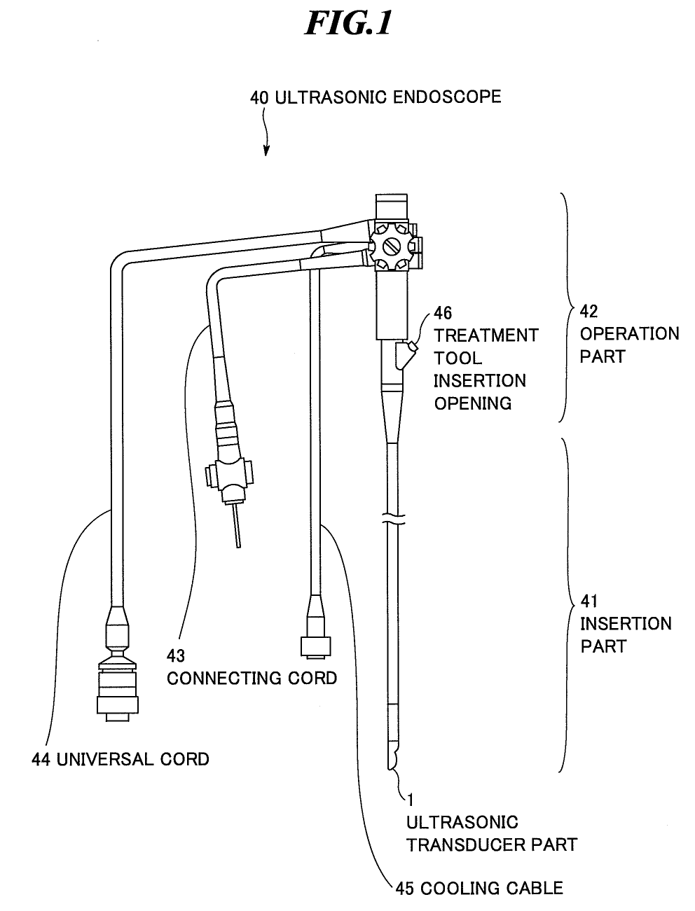

FIG. 1 is a schematic diagram showing an appearance of an ultrasonic endoscope according to the respective embodiments of the present invention. As shown in FIG. 1, an ultrasonic endoscope 40 includes an insertion part 41, an operation part 42, a connecting cord 43, a universal cord 44, and a cooling cable 45. The insertion part 41 includes an elongated tube formed of a member having flexibility for insertion into the body of the object, and an ultrasonic transducer part 1 at the leading end thereof.

The operation part 42 is provided at the base end of the insertion part 41 and connected to an ultrasonic endoscopic apparatus main body via the connecting cord 43, the universal cord 44, and the cooling cable 45. A treatment tool insertion opening 46 provided in the operation part 42 is a hole for leading in a treatment tool such as a punctuation needle or forceps. Various treatments are performed within a body cavity of an object to be inspected by operating it with the operation part 42.

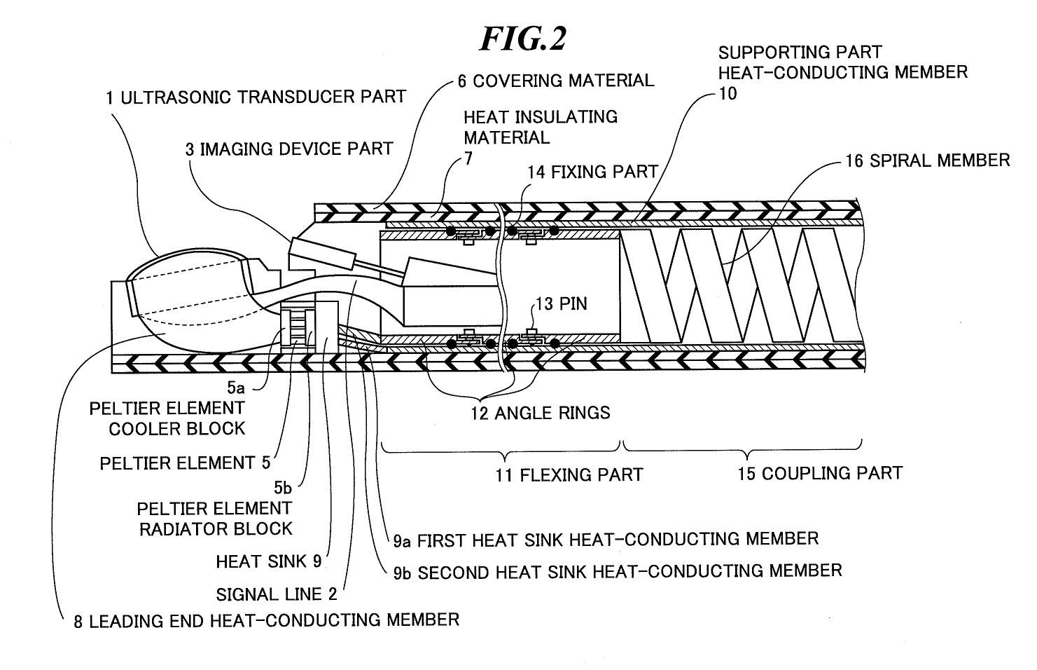

FIG. 2 shows the leading end of the insertion part of the ultrasonic endoscope according to the first embodiment of the present invention. As shown in FIG. 2, the leading end of the insertion part of the ultrasonic endoscope according to the first embodiment has an ultrasonic transducer part 1 for transmitting and receiving ultrasonic waves, a signal line 2 for transmitting signals between the ultrasonic transducer part 1 and the ultrasonic endoscope main body, an imaging device part 3 for optically imaging affected parts, a Peltier element 5 for cooling the ultrasonic transducer part 1, a flexing part 11 that is flexible and flexibly supports the ultrasonic transducer part 1, the imaging device part 3, and the Peltier element 5, a coupling part 15 for coupling the flexing part 11 to the operation part 42 (FIG. 1), a covering material 6 for covering at least the flexing part 11 and the coupling part 15, and a heat (thermal) insulating material 7 provided inside of the covering material 6 at least in the flexing part 11 and the coupling part 15, for thermally insulating the inside from outside.

The ultrasonic transducer part 1 has 64 ultrasonic transducers arranged on a backing material, for example. The signal line 2 includes plural shield lines connected to the 64 ultrasonic transducers, respectively, for example. The flexing part 11 includes plural angle rings 12 connected to one another to be relatively displaced by pins 13. The coupling part 15 includes a spiral member 16. The spiral member 16 is generally formed of stainless steel, but, in the embodiment, preferably formed of copper or copper alloy in view of heat release. The covering material 6 is formed of an insulating material such as fluorine-containing rubber. The heat insulating material 7 is formed of a heat insulating material such as glass fiber, electron beam cross-linking polyolefin foam, or phenol foam. The heat insulating material 7 may be formed by shaping the heat insulating material in a sheet shape.

The Peltier element 5 for cooling the ultrasonic transducer part 1 has a joining part formed of P-type and N-type heterogeneous semiconductors or heterogeneous metals, a cooler block 5a provided on one end of the joining part, and a radiator block 5b provided on the other end of the joining part. When current is flowed in the joining part in a predetermined direction, cooling is performed on the cooling surface of the cooler block 5a and heat release is performed on the radiating surface of the radiator block 5b.

A leading end heat-conducting member 8 is provided for reducing the thermal resistance between the ultrasonic transducer part 1 and the cooler block 5 and effectively transferring heat. The leading end heat-conducting member 8 has high heat conductivity and flexibility and durability to bending, and is formed in a foil, wire, mesh, or sheet shape having a thickness of 30 μm to several hundreds of micrometers by employing a material including metal and/or graphite. Preferably, the metal material includes copper or copper alloy with good heat conductivity. Since the leading end heat-conducting member 8 is formed in a thin layer, the ultrasonic transducer part 1 and the leading end heat-conducting member 8 can be accommodated within the endoscope tube having a small diameter.

One end of the leading end heat-conducting member 8 is connected to the side surface of the ultrasonic transducer part 1, and the other end of the leading end heat-conducting member 8 is connected to the cooler block 5a provided on the cooling surface of the Peltier element 5. The radiating surface of the radiator block 5b is connected to one end of a heat sink 9. The other end of the heat sink 9 may be connected to a supporting part heat-conducting member 10 by employing a first heat sink heat-conducting member 9a, such that the heat generated in the ultrasonic transducer part 1 is transferred to the supporting part heat-conducting member 10 via the heat sink 9. Alternatively, the other end of the heat sink 9 may be connected to the angle rings 12 in contact with the supporting part heat-conducting member 10 by employing a second heat sink heat-conducting member 9b, such that the heat generated in the ultrasonic transducer part 1 is transferred from the heat sink 9 further to the supporting part heat-conducting member 10 via the angle rings 12 of the flexing part 11.

In the ultrasonic endoscope according to the embodiment, at least in the flexing part 11 and the coupling part 15, the supporting part heat-conducting member 10 is provided at the inner side of the heat insulating material 7 provided inside of the covering material 6. The supporting part heat-conducting member 10 may be extended to a part of the operation part 42 (FIG. 1). The supporting part heat-conducting member 10 has high heat conductivity and flexibility and durability to bending, and is formed in a foil, wire, mesh, or sheet shape by employing a material including metal and/or graphite. Preferably, the metal material includes copper or copper alloy with good heat conductivity. Thereby, heat is released from the leading end of the insertion part 41 (FIG. 1) toward the operation part 42. In the ultrasonic endoscope according to the embodiment, the Peltier element 5 is provided, and thus, heat release from the ultrasonic transducer part 1 to the Peltier element 5 is promoted and the temperature rise in the ultrasonic transducer part 1 is suppressed. However, since the Peltier element 5 is provided, the heat is released more from the insertion part 41 to the operation part 42 than in the case without the Peltier element 5. Further, since the heat insulating material 7 is provided inside of the covering material 6, heat release to the outside air via the covering material 6 is also suppressed. Under the condition, heat is released from the Peltier element 5 toward the operation part 42 by the supporting part heat-conducting member 10.

Further, the supporting part heat-conducting member 10 may be provided to also serve as a shield layer inside of the covering material 6. Furthermore, no shield layer may be provided but the supporting part heat-conducting member 10 may be provided inside of the covering material 6 for reducing the diameter of the insertion part of the ultrasonic endoscope. The supporting part heat-conducting member 10 may be electrically insulated from the ground line of the ultrasonic endoscope main body so as to reduce the inductive noise from the ultrasonic endoscope main body.

The flexing part 11 is provided near the leading end of the endoscope, for example. The flexing part 11 is configured by arranging supporting points for bending the plural top-like angle rings 12 with displacement of 90° with respect to each other in a staggered manner. Those angle rings 12 are connected to one another to be relatively displaced by pins 13 and form a hinge structure. Wires are provided inside of the angle rings 12, and the entire flexing part 11 bends and operates like joints. Preferably, the angle rings 12 are formed of a high heat-conducting material such as copper or copper alloy.

However, in the part of the pins 13 connecting the plural angle rings 12, heat is conducted via the pins 13 and sufficient heat release is hardly achieved. On this account, in the embodiment, the angle rings 12 are fixed to the supporting part heat-conducting member 10 having flexibility by fixing parts 14, and heat is released to the supporting part heat-conducting member 10 via the fixing parts 14. The fixing parts 14 may be fixed by soldering, or using metal rings, for example.

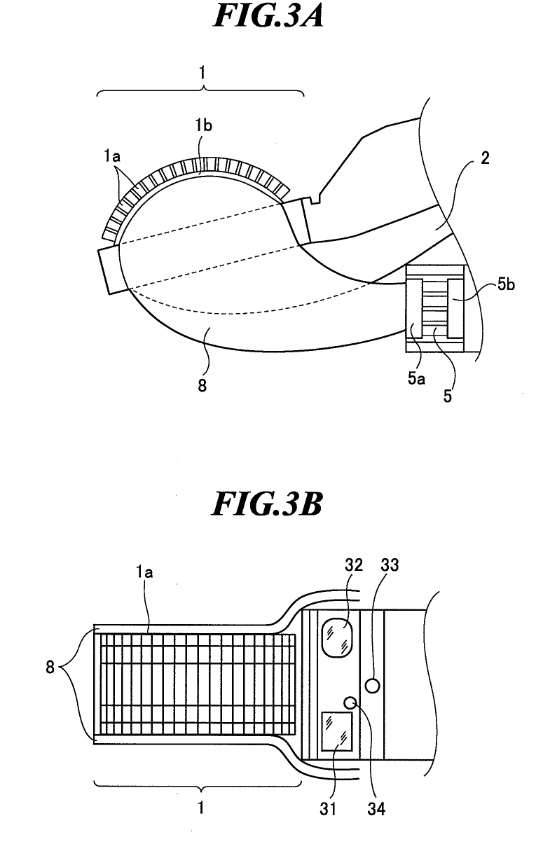

FIGS. 3A and 3B show a configuration around the ultrasonic transducer part in the insertion part of the ultrasonic endoscope shown in FIG. 2. FIG. 3A is a side view of the periphery of the ultrasonic transducer part, and FIG. 3B is a plan view of the periphery of the ultrasonic transducer part.

As shown in FIG. 3B, in the insertion part of the ultrasonic endoscope, the ultrasonic transducer part 1, an observation window 31, an illumination window 32, a treatment tool passage opening 33, and a nozzle hole 34 are provided. In FIG. 3B, an objective lens is fit in the observation window 31, and a solid-state image sensor such as a CCD camera or an input end of an image guide is provided in the imaging position of the objective lens. These configure an observation optics. Further, an illumination lens for outputting illumination light to be supplied from the light source unit via a light guide is fit in the illumination window 32. These configure an illumination optics.

The treatment tool passage opening 33 is a hole for leading out a treatment tool or the like. Various treatments are performed within a body cavity of the object by projecting the treatment tool such as the punctuation needle or forceps (not shown) from the hole. The nozzle hole 34 is provided for injecting a liquid (water or the like) for cleaning the observation window 31 and the illumination window 32.

The ultrasonic transducer part 1 is a convex-type multi row array and includes plural ultrasonic transducers 1a arranged in five rows, for example, and a backing material 1b on which those ultrasonic transducers 1a are arranged. The leading end heat-conducting member 8 may be joined to the side surface of the backing material 1b. Alternatively, the leading end heat-conducting member 8 may be joined to the side surface of the backing material 1b and the side surfaces of the plural ultrasonic transducers 1a in the ultrasonic transducer part 1. In this case, it is necessary to electrically insulate the individual electrodes of the ultrasonic transducers 1a from the leading end heat-conducting member 8. A common electrode of the ultrasonic transducers 1a may be connected to the leading end heat-conducting member 8 or electrically insulated from the leading end heat-conducting member 8.

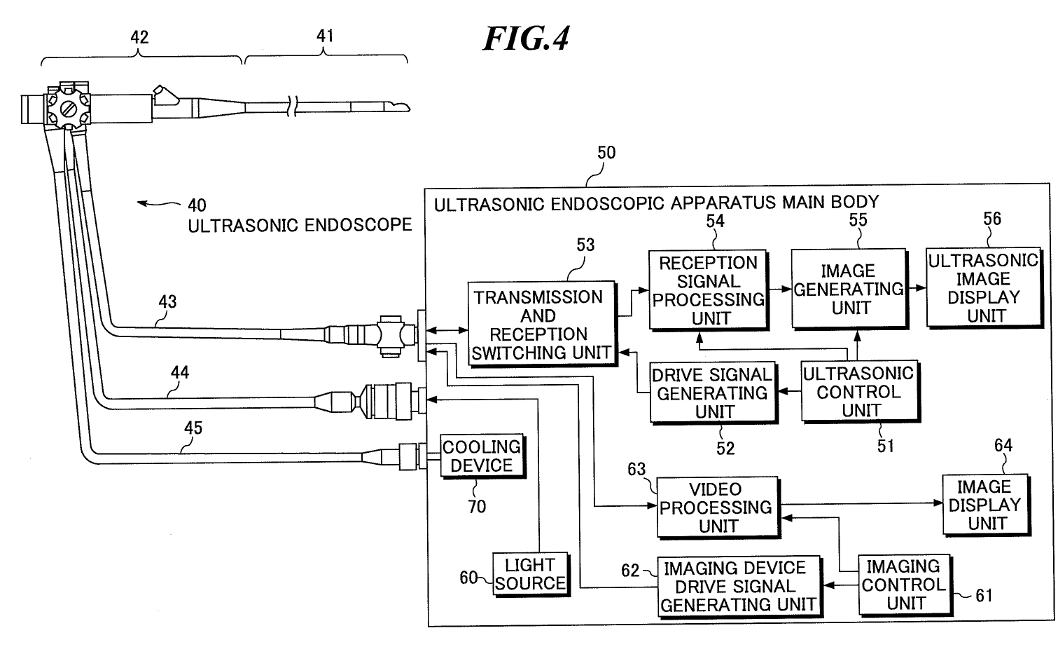

FIG. 4 shows an ultrasonic endoscopic apparatus including the ultrasonic endoscope according to the respective embodiments of the present invention and the ultrasonic endoscopic apparatus main body. The plural ultrasonic transducers included in the ultrasonic transducer part 1 (FIG. 2) are electrically connected to the ultrasonic endoscopic apparatus main body 50 by the plural shield lines via the insertion part 41, the operation part 42, and the connecting cord 43. Those shield lines transmit plural drive signals generated in the ultrasonic endoscopic apparatus main body 50 to the respective ultrasonic transducers and transmit plural reception signals outputted from the respective ultrasonic transducers to the ultrasonic endoscopic apparatus main body 50.

The ultrasonic endoscopic apparatus main body 50 includes an ultrasonic control unit 51, a drive signal generating unit 52, a transmission and reception switching unit 53, a reception signal processing unit 54, an image generating unit 55, an ultrasonic image display unit 56, a light source 60, an imaging control unit 61, an imaging device drive signal generating unit 62, a video processing unit 63, and an image display unit 64. Further, the ultrasonic endoscopic apparatus main body 50 may include a cooling device 70.

The ultrasonic control unit 51 controls imaging operation using the ultrasonic transducer part 1. The drive signal generating unit 52 includes plural drive circuits (pulsers or the like), for example, and generates plural drive signals to be used for respectively driving the plural ultrasonic transducers. The transmission and reception switching unit 53 switches between output of the drive signals to the ultrasonic transducer part 1 and input of the reception signals from the ultrasonic transducer part 1.

The reception signal processing unit 54 includes plural preamplifiers, plural A/D converters, a digital signal processing circuit or CPU, for example, and performs predetermined signal processing such as amplification, phase adjusting and addition, and detection on the reception signals to be outputted from the plural ultrasonic transducers. The image generating unit 55 generates image data representing ultrasonic images based on the reception signals on which the predetermined signal processing has been performed. The ultrasonic image display unit 56 displays the ultrasonic images based on the image data generated in this manner.

The light source 60 emits light to be used for illumination of the object. The light outputted from the light source 60 illuminates the object via the universal cord 44 through the illumination window 32 (FIG. 3B) of the insertion part 41. The illuminated object is imaged by the imaging device part 3 through the observation window 31 (FIG. 3B) of the insertion part 41, and video signals outputted from the imaging device part 3 are inputted to the video processing unit 63 of the ultrasonic endoscopic apparatus main body 50 via the connecting cord 43.

The imaging control unit 61 controls imaging operation using the imaging device part 3. The imaging device drive signal generating unit 62 generates drive signals to be supplied to the imaging device part 3. The video processing unit 63 generates image data based on the video signals to be inputted from the imaging device part 3. The image display unit 64 inputs the image data from the video processing unit 63 and displays images of the object.

The cooling device 70 cools the cooling medium to a predetermined temperature and supplies the cooled cooling medium to the operation part 42 of the ultrasonic endoscope via the first tube within the cooling cable 45, so as to cool the supporting part heat-conducting member 10 in the operation part 42. Thereby, the ultrasonic transducer 1 and/or imaging device part 3 (FIG. 2) included in the insertion part 41 are cooled via the supporting part heat-conducting member 10. In the embodiment, the Peltier element 5 is provided for promoting the heat release from the ultrasonic transducer part 1, however, the amount of heat release from the Peltier element to the supporting part heat-conducting member 10 increases, and it may be more necessary to cool the supporting part heat-conducting member 10. The cooling medium heated by the heat generation of the ultrasonic transducer part 1 and/or imaging device part 3 is collected into the cooling device 70 via the second tube within the cooling cable 45 again. In this manner, the cooling medium circulates.

Next, the second embodiment of the present invention will be explained.

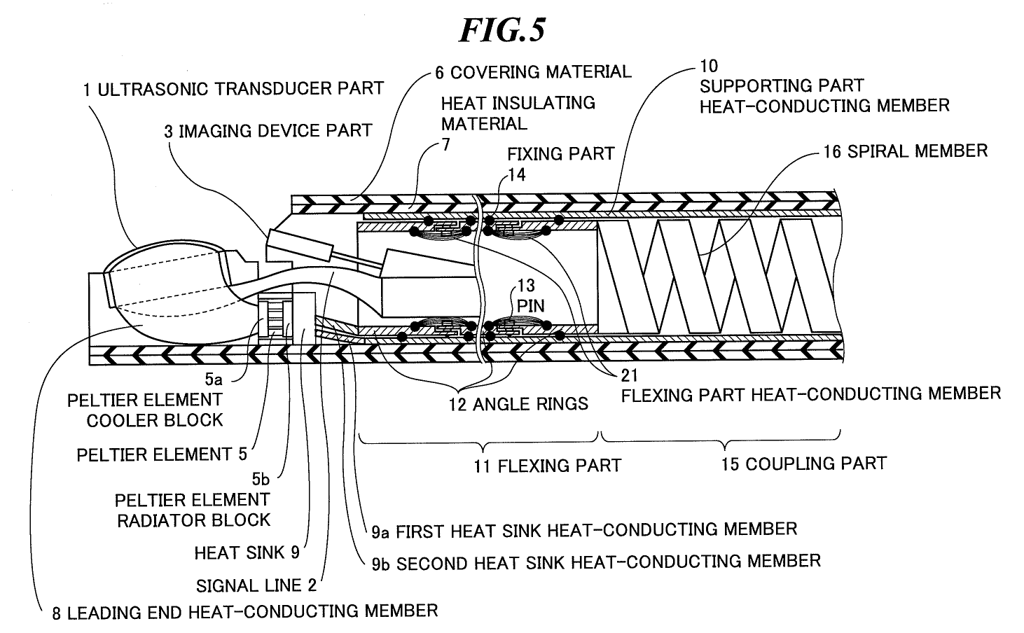

FIG. 5 schematically shows a leading end of an insertion part of an ultrasonic endoscope according to the second embodiment of the present invention. In the second embodiment of the present invention, the plural angle rings 12 of the flexing part 11 are connected by the pins 13 as is the case of the first embodiment of the present invention, but sufficient heat release is hardly achieved through the connection by the pins 13. On this account, heat is released from the plural angle rings 12 to the supporting part heat-conducting member 10 via the fixing parts 14, and a flexing part heat-conducting member 21 is provided for promotion of heat transfer among the angle rings 12.

The flexing part heat-conducting member 21 has high heat conductivity and flexibility and durability to bending, and formed in a foil, wire, mesh, or sheet shape by employing a material including metal and/or graphite. Preferably, the metal material includes copper or copper alloy with good heat conductivity. According to the second embodiment, since heat is transmitted among the plural angle rings 12 via the flexing part heat-conducting member 21, the heat is released to the supporting part heat-conducting member 10 more efficiently than in the case of the first embodiment.

Next, the third embodiment of the present invention will be explained.

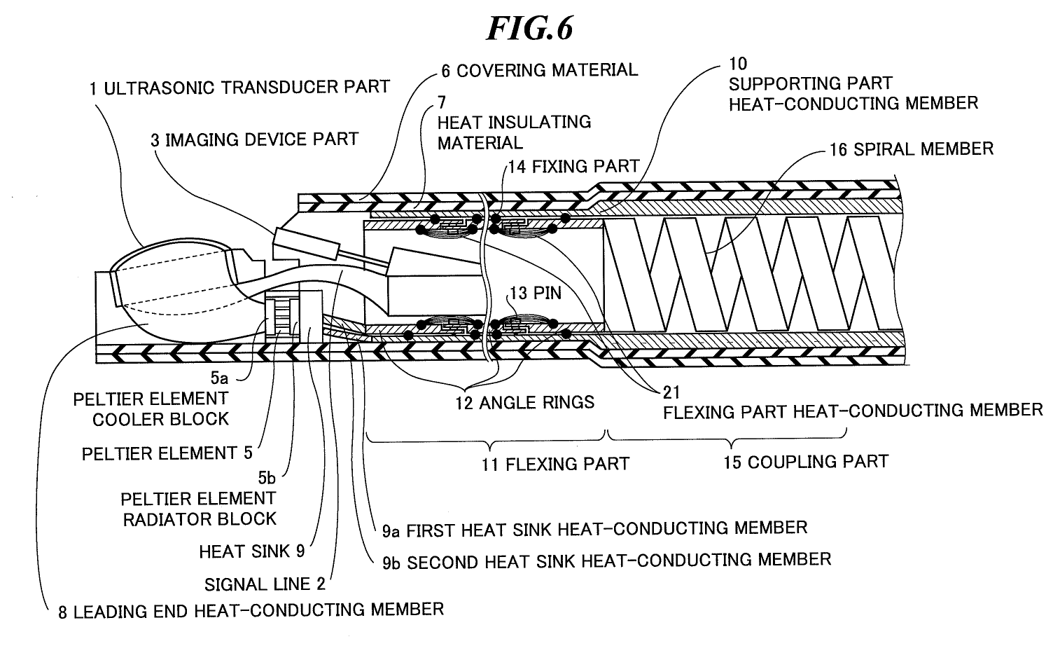

FIG. 6 schematically shows a leading end of an insertion part of an ultrasonic endoscope according to the third embodiment of the present invention. In the third embodiment of the present invention, the outer diameter of a part from the ultrasonic transducer part 1 to the flexing part 11 is made smaller than the outer diameter of the coupling part 15 nearer the operation part 42 (FIG. 1) side than the flexing part 11. Here, the flexing part 11 is not gradually thin but uniformly thin, and thus, its insertability into thin bronchial tubes is good. Further, the sectional area of the supporting part heat-conducting member 10 at the coupling part 15 is made larger than the sectional area of the supporting part heat-conducting member 10 at the flexing part 11. Thereby, heat radiation of the coupling part 15, which becomes insufficient when the ultrasonic transducers are stacked for increasing the transmission output of ultrasonic waves and/or the Peltier element is provided and the amount of heat release from the Peltier element increases, is improved and both the greater output and the heat radiation can be achieved.

The flexing part heat-conducting member 21 that has been explained in the second embodiment may be added to the third embodiment. By adding the flexing part heat-conducting member 21, heat is transferred among the plural rings 12 via the flexing part heat-conducting member 21, and thus, the heat is released to the supporting part heat-conducting member 10 more efficiently.

1. An ultrasonic endoscope comprising:
an ultrasonic transducer part having plural ultrasonic transducers for transmitting and receiving ultrasonic waves;
a Peltier element having a cooling surface and a radiating surface, the cooling surface coupled to said ultrasonic transducer part;
a flexing part that is flexible and supports said ultrasonic transducer part and said Peltier element;
a coupling part that couples said flexing part to an operation part;
a covering material that covers at least said flexing part and said coupling part;
a thermal insulating material that is provided inside of said covering material and thermally insulates at least a part from the radiating surface of said Peltier element to said operation part from outside; and
a heat-conducting member that is provided inside of said thermal insulating material and coupled to the radiating surface of said Peltier element, and transfers heat generated in said ultrasonic transducer part to said operation part.

2. The ultrasonic endoscope according to claim 1, further comprising:
an imaging device part that optically images an object to be inspected.

3. The ultrasonic endoscope according to claim 1, wherein said thermal insulating material includes one of glass fiber, electron beam cross-linking polyolefin foam, and phenol foam.

4. The ultrasonic endoscope according to claim 1, wherein said heat-conducting member includes metal and/or graphite.

5. The ultrasonic endoscope according to claim 1, further comprising:
a second heat-conducting member that is connected to a side surface of said ultrasonic transducer part and the cooling surface of said Peltier element, and transfers the heat generated in said ultrasonic transducer part to said cooling surface of said Peltier element.

6. The ultrasonic endoscope according to claim 5, wherein said second heat-conducting member includes metal and/or graphite.

7. The ultrasonic endoscope according to claim 1, wherein said flexing part includes plural angle rings and plural pins for connecting said plural angle rings to one another to be relatively displaced, and at least one of said plural angle rings is coupled to said Peltier element and said heat-conducting member to transfer the heat generated in said ultrasonic transducer part from said Peltier element to said heat-conducting member.

8. The ultrasonic endoscope according to claim 7, further comprising:
plural fixing parts that fix said plural angle rings to said heat-conducting member and transfer the heat generated in said ultrasonic transducer part from said plural angle rings to said heat-conducting member.

9. The ultrasonic endoscope according to claim 7, further comprising:
a third heat-conducting member connected between adjacent two angle rings.

10. The ultrasonic endoscope according to claim 9, wherein said third heat-conducting member includes metal and/or graphite.

11. The ultrasonic endoscope according to claim 1, wherein an outer diameter of a part from said ultrasonic transducer part to said flexing part is smaller than that of a part from said coupling part to said operation part.

12. The ultrasonic endoscope according to claim 1, wherein a sectional area of said heat-conducting member at said coupling part is larger than that of said heat-conducting member at said flexing part.

13. An ultrasonic endoscopic apparatus comprising:
an ultrasonic endoscope including an ultrasonic transducer part having plural ultrasonic transducers for transmitting and receiving ultrasonic waves, a Peltier element having a cooling surface and a radiating surface, the cooling surface coupled to said ultrasonic transducer part, a flexing part that is flexible and supports said ultrasonic transducer part and said Peltier element, a coupling part that couples said flexing part to an operation part, a covering material that covers at least said flexing part and said coupling part, a thermal insulating material that is provided inside of said covering material and thermally insulates at least a part from the radiating surface of said Peltier element to said operation part from outside, and a heat-conducting member that is provided inside of said thermal insulating material and coupled to the radiating surface of said Peltier element, and transfers heat generated in said ultrasonic transducer part to said operation part; and
an ultrasonic endoscopic apparatus main body that processes signals from said ultrasonic endoscope to display ultrasonic images, and includes a cooing device for cooling said heat-conducting member.
